(19) United States
(12) Patent Application Publication
(10) Pub. No.: US 2020/0237391 A1
(43) Pub. Date: Jul. 30, 2020

(54) MEDICAL DEVICE, METHOD OF MANUFACTURING MEDICAL DEVICE, AND METHOD OF ADJUSTING MEDICAL DEVICE

(71) Applicant: OLYMPUS CORPORATION, Tokyo (JP)

(72) Inventor: Ojiro KITAMURA, Hachioji-shi (JP)

(73) Assignee: OLYMPUS CORPORATION, Tokyo (JP)

(21) Appl. No.: 16/848,927

(22) Filed: Apr. 15, 2020

Related U.S. Application Data
(63) Continuation of application No. PCT/JP2017/037372, filed on Oct. 16, 2017.

(51) Int. Cl. A61B 17/29; A61B 34/00; B23K 26/22

(52) U.S. Cl. CPC A61B 17/2909; A61B 2017/00526; B23K 26/22; A61B 34/71

(57) ABSTRACT

According to one embodiment, a medical device includes: a sheath with a distal end and a proximal end; a housing provided at the proximal end of the sheath; an end effector provided at the distal end of the sheath, the end effector capable of flexing or curving with respect to the sheath; a flexural drive unit including a drive member configured to move relative to the sheath and thereby flex or curve the end effector with respect to the sheath; and a support member mounted inside the housing with a part of the flexural drive unit mounted inside the support member.

### CROSS-REFERENCE TO RELATED APPLICATIONS

This is a Continuation Application of PCT Application No. PCT/JP2017/037372, filed Oct. 16, 2017, the entire contents of which are incorporated herein by reference.

### FIELD

Exemplary embodiments relate to a medical device configured so that an end effector for treating a treatment target flexes or curves with respect to a sheath, a method of manufacturing the medical device, and a method of adjusting the medical device.

### BACKGROUND

U.S. Patent Application Publication No. 2012/0074200A discloses a medical device in which an end effector is installed such that the end effector can curve (flex) with respect to the sheath. The medical device is provided with a drive member connecting between an operation member to which an operation for flexing the end effector is input and the end effector. When the drive member moves in accordance with the operation input to the operation member, the end effector curves (flexes) with respect to the sheath.

This medical device can require a position at which the drive member is connected to the housing to be adjustable when mounting the drive member between the end effector and the operation section.

### SUMMARY

A medical device can include a sheath with a distal end and a proximal end; a housing provided at the proximal end of the sheath; an end effector provided at the distal end of the sheath, the end effector capable of flexing or curving with respect to the sheath; a flexural drive unit including a drive member configured to move relative to the sheath and thereby flex or curve the end effector with respect to the sheath; and a support member mounted inside the housing with a part of the flexural drive unit mounted inside the support member.

### BRIEF DESCRIPTION OF THE DRAWINGS

FIG. 1 is a schematic view of a medical device according to an exemplary embodiment.

FIG. 2 is a schematic perspective view of a configuration of an end effector according to an exemplary embodiment.

FIG. 3 is a schematic view of an internal configuration of a housing according to an exemplary embodiment, observed at a cross section along a longitudinal axis.

FIG. 4 is a schematic perspective view of the internal configuration of the housing according to an exemplary embodiment.

FIG. 5 is a schematic view of a connected structure of a first connector and a second connector according to an exemplary embodiment, observed at a cross section along the longitudinal axis.

FIG. 6A is a cross-sectional view taken along line A-A in FIG. 5 according to an exemplary embodiment.

FIG. 6B is a cross-sectional view taken along line A-A in FIG. 5 according to another exemplary embodiment.

FIG. 7 is a schematic perspective view of a manner of connecting a wire to a first rotary body when assembling the medical device according to an exemplary embodiment.

FIG. 8 is a schematic perspective view of a state in which the wire is mounted to the first rotary body when assembling the medical device according to an exemplary embodiment.

FIG. 9 is a schematic perspective view of a manner of mounting a support member to the first rotary body when assembling the medical device according to an exemplary embodiment.

FIG. 10 is a schematic perspective view of a state in which the support member is mounted to the first rotary body when assembling the medical device according to an exemplary embodiment.

FIG. 11 is a schematic view of a state in which the wire is loosened when assembling the medical device according to an exemplary embodiment.

FIG. 12 is a schematic view of a manner of bonding the support member to the housing with a predetermined tension applied to the wire when assembling the medical device according to an exemplary embodiment.

FIG. 13 is a schematic view of an internal configuration of a housing according to another exemplary embodiment, observed at a cross section along a longitudinal axis.

### DETAILED DESCRIPTION

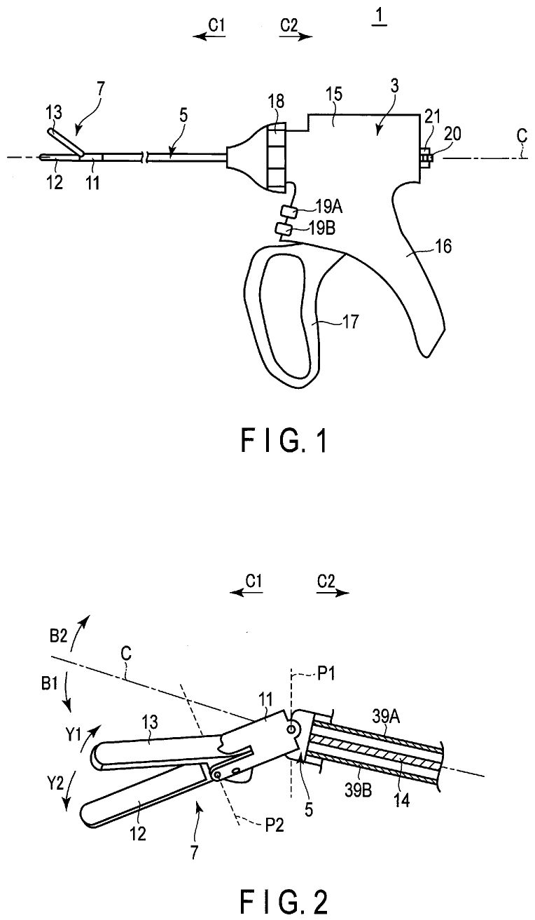

FIG. 1 shows a configuration of a treatment instrument 1, which is a medical device of the present embodiment. As shown in FIG. 1, the treatment instrument 1 includes a holdable housing 3 and a tubular sheath (outer pipe) 5 connected to the housing 3. The sheath 5 defines a longitudinal axis C. The direction along the longitudinal axis C is defined as a longitudinal direction. One side in the longitudinal direction is defined as a distal side (arrow C1 side in FIG. 1), and the opposite side from the distal side is defined as a proximal side (arrow C2 side in FIG. 1). The sheath 5 extends from the proximal side to the distal side along the longitudinal axis C, and is connected to the distal side of the housing 3.

The housing 3 includes a housing main body (handle) 15, which extends along the longitudinal axis C, and a rotation operation knob 18 mounted to the distal end of the housing main body 15. The rotation operation knob 18 is mounted rotatably about the longitudinal axis C with respect to the housing main body 15. The sheath 5 is inserted from the distal side into the rotation operation knob 18 and fixed to the rotation operation knob 18. An end effector 7 for treating a treatment target is mounted to the distal side of the sheath 5. By rotating the rotation operation knob 18 about the longitudinal axis C with respect to the housing main body 15, the operation of rotating the end effector 7 about the longitudinal axis C is input to the rotation operation knob 18. When an operation input is performed to the rotation operation knob 18, a drive force (rotational drive force) is transmitted to the sheath 5, so that the rotation operation knob 18 and the sheath 5 rotate together about the longitudinal axis C with respect to the housing main body 15.

FIG. 2 shows a configuration of the end effector 7. The end effector 7 includes an effector base 11 mounted to the sheath 5, a first grasping piece 12 fixed to the effector base 11, and a second grasping piece 13 pivotably connected to the effector base 11. The effector base 11 is mounted to the sheath 5 such that the effector base 11 is rotatable about a rotation axis (flexure rotation axis) P1 with respect to the sheath 5. The rotation axis P1 extends along a direction intersecting with (perpendicular or approximately perpendicular to) the longitudinal direction of the sheath 5. When the end effector 7 including the effector base 11 rotates about the rotation axis P1 with respect to the sheath 5, the end effector 7 flexes with respect to the sheath 5 in the directions indicated by arrow B1 and arrow B2 in FIG. 2. In another example, a plurality of curving pieces are juxtaposed between the sheath 5 and the end effector 7 in a longitudinal direction, so that the end effector 7 can curve with respect to the sheath 5.

The second grasping piece 13 is pivotable about a rotation axis (opening/closing rotation axis) P2 with respect to the effector base 11. The rotation axis P2 extends along a direction intersecting with (perpendicular or approximately perpendicular to) the longitudinal direction and also intersecting with (perpendicular or approximately perpendicular to) the direction in which the rotation axis P1 extends. When the second grasping piece 13 pivots about the rotation axis P2, the first grasping piece 12 and the second grasping piece 13 are opened or closed with respect to each other in the end effector 7. More specifically, when the second grasping piece 13 pivots, the end effector 7 opens or closes in the directions indicated by arrow Y1 and arrow Y2 in FIG. 2. Both the first grasping piece 12 and the second grasping piece 13 may be mounted pivotably with respect to the effector base 11 (e.g., about the rotation axis P2). In this case, the first grasping piece 12 and the second grasping piece 13 are rotated, so that the first grasping piece 12 and the second grasping piece 13 are opened or closed with respect to each other, and the end effector 7 opens or closes. By the closing of the first grasping piece 12 and the second grasping piece 13 with respect to each other, a treatment target, such as a body tissue, can be grasped between the first grasping piece 12 and the second grasping piece 13.

As shown in FIG. 1, the housing 3 includes a grip (fixed handle) 16, which extends from the housing main body 15 in a direction away from the longitudinal axis C. The direction intersecting with (perpendicular or approximately perpendicular to) the longitudinal axis C and intersecting with (perpendicular or approximately perpendicular to) the direction in which the grip 16 extends is defined as a width direction of the housing 3. The sheath 5 is connected to the housing main body 15 from the distal side. A movable handle 17 is pivotably mounted to the housing main body 15. The movable handle 17 is positioned on the side where the grip 16 is positioned with respect to the longitudinal axis C, and positioned on the distal side with respect to the grip 16 in the present embodiment. By pivoting with respect to the housing main body 15, the movable handle 17 opens or closes with respect to the grip 16. When the movable handle 17 opens or closes with respect to the grip 16, the operation for opening or closing the end effector 7 as described above is input to the movable handle 17, which is an opening/closing operation input unit. The movable handle 17 and the second grasping piece 13 are connected to each other via a movable member 14 extending in the sheath 5 along the longitudinal axis C. By opening or closing the movable handle 17, which is an opening/closing operation input unit, with respect to the grip 16, the movable member 14 moves along the longitudinal axis C with respect to the sheath 5 and the housing 3, and the second grasping piece 13 pivots about the rotation axis P2. As a result, the pair of grasping pieces 12 and 13 open or close with respect to each other.

Also, the movable member 14 is rotatable about the longitudinal axis C together with the sheath 5. Hence, in response to the operation input performed to the rotation operation knob 18, the end effector 7 rotates about the longitudinal axis C with respect to the housing main body 15 together with the sheath 5 and the movable member 14.

Operation buttons 19A and 19B, which are energy operation input units, are mounted to the housing main body 15 of the housing 3. When an operation input is performed to the operation button 19A, a high-frequency electric energy is supplied to the grasping pieces 12 and 13, for example. Then, a high-frequency current is applied to the treatment target held between the grasping pieces 12 and 13, thereby treating the treatment target. When an operation input is performed to the operation button 19B, electric energy is supplied to a heating element (not shown) provided to the end effector 7, for example. Then, the heat generated by the heating element is used to treat the treatment target. The energy supplied to the end effector 7 is not limited to the aforementioned energy. It suffices that energy used for treatment is supplied to the end effector 7 in response to the operation input performed to the operation buttons 19A and 19B.

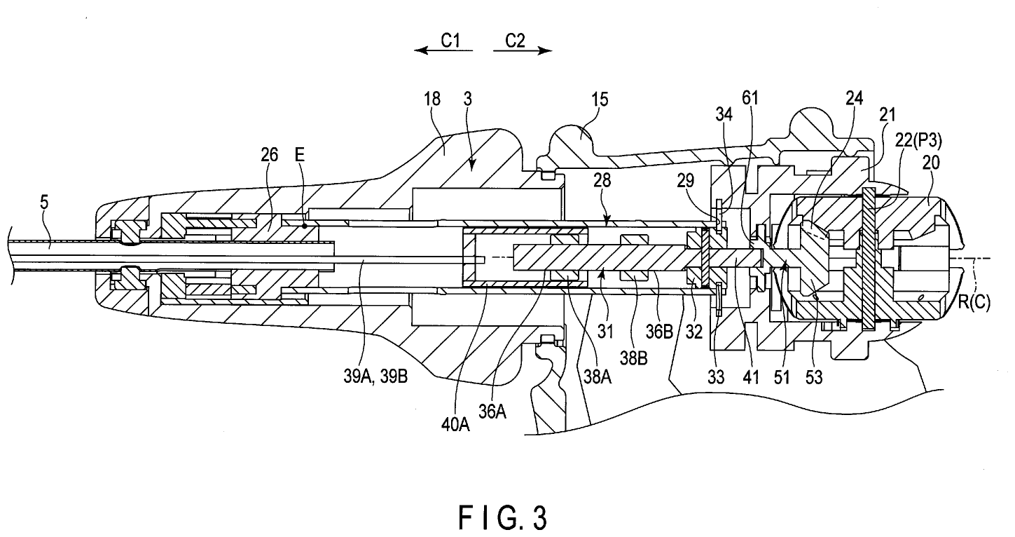

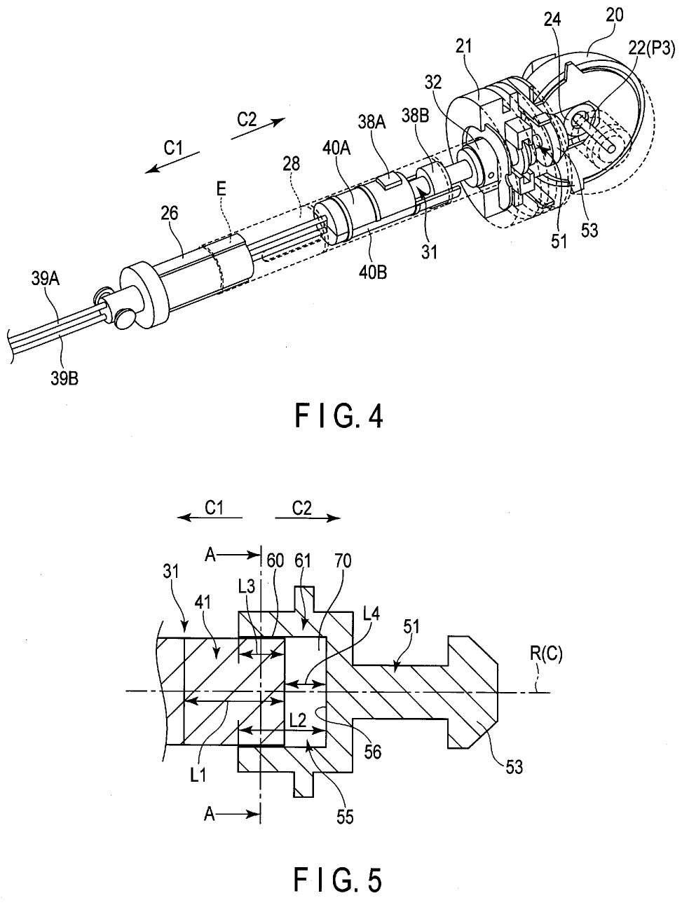

FIGS. 3 and 4 are diagrams showing the internal configuration of the housing 3. As shown in FIGS. 3 and 4, a holder (base member) 21, a rotary base 26, and a support member 28 are provided inside the housing 3.

The holder 21 is a tubular member extending along the longitudinal axis C. The central axis of the holder 21 is coincident or approximately coincident with the longitudinal axis C. The holder 21 is supported by the housing main body 15 at the proximal end of the housing main body 15. The holder 21 is rotatable about the longitudinal axis C with respect to the housing main body 15.

The rotary base 26 is a tubular member extending along the longitudinal axis C. The central axis of the rotary base 26 is coincident or approximately coincident with the longitudinal axis C. The distal end of the rotary base 26 is fixed to the inside of the rotation operation knob 18. When the rotation operation knob 18 rotates with respect to the housing main body 15, the rotary base 26 rotates about the longitudinal axis C with respect to the housing main body 15 together with the rotation operation knob 18.

The support member 28 extends along the longitudinal axis C. The support member 28 is formed in a tubular shape. The central axis of the support member 28 is coincident or approximately coincident with the longitudinal axis C. The distal end of the support member 28 is bonded to the proximal end of the rotary base 26. By being bonded to the rotary base 26, the support member 28 is fixed to the rotation operation knob 18. The proximal end of the support member 28 is fixed to the holder 21.

When the rotation operation knob 18 rotates about the longitudinal axis C, the rotary base 26, the support member 28, and the holder 21 rotate about the longitudinal axis C with respect to the housing main body 15 together with the rotation operation knob 18.

A flexural drive unit configured to flex the end effector 7 with respect to the sheath 5 is formed inside the housing 3 and inside the sheath 5. In the present embodiment, the flexural drive unit is formed of an operation dial (flexural operation dial) 20, a first rotary body (first rotor) 31, a second rotary body (second rotor) 51, and wires (drive members) 39A and 39B.

The operation dial 20 is mounted to the holder 21 via a connecting pin 22. The connecting pin 22 extends along a central axis P3. The central axis P3 extends along a direction intersecting with (perpendicular or approximately perpendicular to) the longitudinal axis C. The operation dial 20 is rotatable about the central axis P3 with respect to the holder 21. By rotating the operation dial 20 about the central axis P3 with respect to the holder 21, the operation of rotating (flexing) the end effector 7 with respect to the sheath 5 is input to the operation dial 20. The operation dial 20 is an operation member to which the operation of flexing or curving the end effector 7 with respect to the sheath 5 is input.

The operation dial 20 includes a gear section 24. The gear section 24 is provided over the entire circumference about the central axis P3.

The rotary body 51 extends along a rotation axis R. In the present embodiment, the rotation axis R of the rotary body 31 is coincident or approximately coincident with the longitudinal axis C. The rotary body 51 is mounted to the holder 21 and rotates about the longitudinal axis C with respect to the housing main body 15 together with the holder 21. Also, the rotary body 51 is rotatable about the rotation axis R with respect to the holder 21 and the operation dial 20. The rotary body 51 is supported by the holder 21 with the movement of the rotary body 51 relative to the holder 21 and the operation dial 20 in the longitudinal direction regulated.

The rotary body 51 includes a gear section 53. The gear section 53 is provided at the proximal end of the rotary body 51. A gear is formed on the outer peripheral surface of the gear section 53 about the rotation axis R (the longitudinal axis C in the present embodiment). The gear formed on the gear section 53 is, for example, a bevel gear (an umbrella gear).

The gear section 53 meshes (engages) with the gear section 24 of the operation dial 20. When the operation dial 20 rotates about the central axis P3 with respect to the holder 21, the drive force is transmitted via the engagement between the gear section 24 and the gear section 53, causing the rotary body 51 to rotate about the rotation axis R with respect to the holder 21.

The rotary body 31 extends along the rotation axis R and is formed in an approximately rod shape. The rotary body 31 is mounted inside the support member 28. The proximal end of the rotary body 31 projects from the inside of the support member 28 to the proximal side. The rotary body 31 is, for example, a ball screw.

The rotary body 31 is arranged on the distal side with respect to the rotary body 51. The rotary body 31 is juxtaposed with the rotary body 51 along the rotation axis R. The proximal end of the rotary body 31 and the distal end of the rotary body 51 are connected to each other. The rotation of the rotary body 31 about the rotation axis R with respect to the rotary body 51 is regulated. Therefore, when the rotary body 51 rotates about the rotation axis R with respect to the holder 21, the drive force (rotational drive force) is transmitted from the rotary body 51 to the rotary body 31, so that the rotary body 31 and the rotary body 51 rotate together about the rotation axis R with respect to the holder 21 and the housing 3.

A ring member 32 is mounted to the rotary body 31. The ring member 32 is formed in a ring shape and fixed to the outside of the rotary body 31. An engaging groove 33 extending about the rotation axis R is formed on the outer peripheral surface of the ring member 32. The engaging groove 33 is a groove recessed inwardly with respect to the outer peripheral surface of the ring member 32. The engaging groove 33 is provided over the entire circumference about the rotation axis R.

A slit 29 corresponding to the engaging groove 33 of the ring member 32 is formed in the support member 28. The slit 29 is a through-hole passing through the support member 28 from the inside to the outside. In the present embodiment, two slits 29 are provided in the support member 28 and on opposite sides with respect to each other with the rotation axis R interposed therebetween.

With the engaging groove 33 and the slit 29 arranged at about the same position in a direction along the rotation axis R, an engaging member 34 is mounted to the engaging groove 33 and the slit 29, so that the rotary body 31 is mounted to the support member 28. At this time, the rotary body 31 is connected to the support member 28 by the engaging member 34 in such a manner that the rotary body 31 is rotatable about the rotation axis R with respect to the support member 28. As the rotary body 31 is connected to the support member 28 by the engaging member 34, the movement of the rotary body 31 in a direction along the rotation axis R relative to the support member 28 is regulated. Thereby, the rotary body 31 is mounted inside the housing main body 15 while being rotatable about the rotation axis R with respect to the support member 28 and the housing 3, and with the movement of the rotary body 31 relative to the support member 28 and the housing 3 in the direction along the rotation axis R regulated.

The rotary body 31 includes a first screw portion 36A and a second screw portion 36B. The first screw portion 36A and the second screw portion 36B are arranged closer to the distal side as compared to the ring member 32. The first screw portion 36A and the second screw portion 36B are juxtaposed in the direction along the rotation axis R. The first screw portion 36A and the second screw portion 36B have a thread (male thread) formed on the outer periphery of the rotary body 31 about the rotation axis R. The first screw portion 36A and the second screw portion 36B are formed to be reversed with respect to each other.

A pair of nuts (moving members) 38A and 38B are mounted to the rotary body 31. A thread (female thread) to be screwed into the first screw portion 36A is formed on the inner peripheral surface of the first nut 38A. The first nut 38A is mounted to the rotary body 31 by being screwed into the first screw portion 36A. A thread (female thread) to be screwed into the second screw portion 36B is formed on the inner peripheral surface of the second nut 38B. The second nut 38B is mounted to the rotary body 31 by being screwed into the second screw portion 36B.

One end (proximal end) of the first wire 39A is connected to the first nut 38A via a first relay member 40A. The other end (distal end) of the first wire 39A is connected to the effector base 11 of the end effector 7. One end (proximal end) of the second wire 39B is connected to the second nut 38B via a second relay member 40B. The other end (distal end) of the second wire 39B is connected to the effector base 11 of the end effector 7.

Each of the first nut 38A and the second nut 38B is movable relative to the support member 28 in the direction along the rotation axis R. The first nut 38A and the second nut 38B are regulated from rotating about the rotation axis R with respect to the support member 28. Therefore, when the rotary body 31 rotates about the rotation axis R with respect to the support member 28, the first nut 38A and the second nut 38B move in the direction along the rotation axis R (longitudinal axis C in the present embodiment) with respect to the rotary body 31. Since the first screw portion 36A and the second screw portion 36B are reversed with respect to each other, the first nut 38A and the second nut 38B move to opposite sides with respect to each other in the direction along the rotation axis R (longitudinal axis C). When the first nut 38A and the second nut 38B move to opposite sides with respect to each other, one of the first wire 39A and the second wire 39B moves to the proximal side relative to the rotary body 31 and the sheath 5, and the other of the first wire 39A and the second wire 39B moves to the distal side relative to the rotary body 31 and the sheath 5.

With the configuration described above, when the operation dial 20 is rotated about the central axis P3 with respect to the housing main body 15 by the operation input to the operation dial 20, the drive force is transmitted to the rotary body 31 via the rotary body 51, so that the rotary body 31 rotates about the rotation axis R. As described above, the first screw portion 36A and the second screw portion 36B are formed to be reversed with respect to each other. Therefore, when the rotary body 31 rotates about the rotation axis R, the first nut 38A to be screwed into the first screw portion 36A and the second nut 38B to be screwed into the second screw portion 36B move to opposite sides with respect to each other in the direction along the rotation axis R (longitudinal axis C). When the first nut 38A and the second nut 38B move to opposite sides with respect to each other, the first wire 39A connected to the first nut 38A and the second wire 39B connected to the second nut 38B move to opposite sides with respect to each other in the longitudinal direction. When the pair of wires 39A and 39B moves to opposite sides with respect to each other, one of the wires 39A and 39B is pulled and the other of the wires 39A and 39B is loosened. As a result, the end effector 7 flexes toward one side in the flexing direction with respect to the sheath 5.

The wires 39A and 39B are mounted between the end effector 7 and the rotary body 31, and the rotary body 31 is mounted to the rotary base 26 and the rotation operation knob 18 via the support member 28. Therefore, when the rotation operation knob 18 rotates about the longitudinal axis C with respect to the housing main body 15, the rotary base 26, the support member 28, the rotary body 31, the nuts 38A and 38B, the relay members 40A and 40B, and the wires 39A and 39B rotate together about the longitudinal axis C with respect to the housing main body 15.

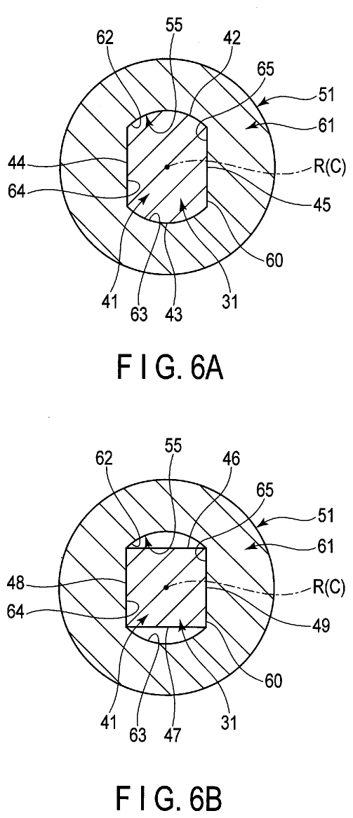

FIGS. 5 and 6A are diagrams showing a connected structure of the rotary body 31 and the rotary body 51. As shown in FIGS. 5 and 6A, the rotary body 31 includes a first connector 41. The first connector 41 is provided at the proximal end of the rotary body 31 and forms a proximal end of the rotary body 31. That is, the first connector 41 forms one end of the rotary body 31 in the direction along the rotation axis R. The cross section of the first connector 41 that intersects (is perpendicular or approximately perpendicular to) the rotation axis R has an irregular shape (first irregular shape).

In the present embodiment, the first connector 41 is formed by the outer peripheral surface of the proximal end of the rotary body 31. The first connector 41 includes two curved sections 42 and 43 extending in parallel or approximately in parallel to the rotation axis R, and two planar sections 44 and 45 extending in parallel or approximately in parallel to the rotation axis R. Each of the curved sections 42 and 43 has an arc shape with the rotation axis R in the center, as viewed in a cross section intersecting (perpendicular or approximately perpendicular to) the rotation axis R. Each of the planar sections 44 and 45 is positioned between the curved sections 42 and 43 about the rotation axis R, that is, in the circumferential direction. Each of the curved sections 42 and 43 is positioned between the planar sections 44 and 45 about the rotation axis R, that is, in the circumferential direction. In the present embodiment, the first connector 41 has two D-cut shapes formed on a cylindrical member.

The rotary body 51 includes a fitting hole 55. The fitting hole 55 is provided at the distal end of the rotary body 51. The fitting hole 55 is a groove that is recessed toward the proximal side on the distal surface of the rotary body 51. That is, the fitting hole 55 is formed on one end face of the rotary body 51 in the direction along the rotation axis R. The fitting hole 55 extends along the rotation axis R. The fitting hole 55 includes a bottom face 56 forming a groove-shaped bottom.

The rotary body 51 includes a second connector 61. The second connector 61 forms a distal end of the rotary body 51. That is, the second connector 61 forms one end of the rotary body 51 in the direction along the rotation axis R. The cross section of the second connector 61 that intersects (is perpendicular or approximately perpendicular to) the rotation axis R has an irregular shape (second irregular shape). The second connector 61 is formed in a shape that fits to the first connector 41. That is, the second connector 61 is formed in a shape complementary to the first connector 41.

In the present embodiment, the second connector 61 is formed by the inner peripheral surface of the fitting hole 55. The second connector 61 includes two curved sections 62 and 63 extending in parallel or approximately in parallel to the rotation axis R, and two planar sections 64 and 65 extending in parallel or approximately in parallel to the rotation axis R. Each of the curved sections 62 and 63 has an arc shape with the rotation axis R in the center, as viewed in a cross section intersecting (perpendicular or approximately perpendicular to) the rotation axis R. Each of the planar sections 64 and 65 is positioned between the curved sections 62 and 63 about the rotation axis R, that is, in the circumferential direction. Each of the curved sections 62 and 63 is positioned between the planar sections 64 and 65 about the rotation axis R, that is, in the circumferential direction.

In the present embodiment, when the proximal end of the rotary body 31 is inserted into the fitting hole 55 of the rotary body 51, the first connector 41 and the second connector 61 engage with each other. At this time, the curved section 42 of the first connector 41 and one of the curved sections of the second connector 61 (for example, the curved section 62) contact each other, and the curved section 43 of the first connector 41 and the other of the curved sections of the second connector 61 (for example, the curved section 63) contact each other. Also, the planar section 44 of the first connector 41 and one of the planar sections of the second connector 61 (for example, the planar section 64) contact each other, and the planar section 45 of the first connector 41 and the other of the planar sections of the second connector 61 (for example, the planar section 65) contact each other. In the present embodiment, the first connector 41 and the second connector 61 are in contact with each other over the entire circumference about the rotation axis R. Therefore, the first connector 41 and the second connector 61 respectively have a contact portion (contact surface or contact point) for each other that is formed over the entire circumference for each other.

By the engagement of the first connector 41 and the second connector 61 with each other, as described above, a fitting portion 60 of the first connector 41 and the second connector 61 is formed. As a result, the rotation of the rotary body 31 about the rotation axis R with respect to the rotary body 51 is regulated, and the rotational drive force about the rotation axis R can be transmitted from the rotary body 51 to the rotary body 31. Therefore, when the rotary body 51 rotates about the rotation axis R, the rotational drive force is transmitted to the rotary body 31, so that the rotary body 31 and the rotary body 51 rotate together about the rotation axis R with respect to the housing main body 15.

It suffices that the first irregular shape of the first connector 41 and the second irregular shape of the second connector 61 are shapes that allow the first connector 41 and the second connector 61 to be engaged with each other so that the rotation with respect to each other is regulated. For example, the first irregular shape and the second irregular shape may be polygonal (such as hexagonal) or elliptical.

In one modification, the first irregular shape may be a rectangular shape that can fit to the second irregular shape, as shown in FIG. 6B. In this case, the first connector 41 includes four planar sections 46, 47, 48, and 49 extending in parallel or approximately in parallel to the rotation axis R. The planar sections 46 and 47 are in parallel or approximately in parallel to each other. The planar sections 48 and 49 are approximately in parallel to each other, and are perpendicular or approximately perpendicular to the planar sections 46 and 47. The first connector 41 and the second connector 61 are fitted to each other. At this time, the planar section 48 of the first connector 41 and one of the planar sections of the second connector 61 (for example, the planar section 64) contact each other, and the planar section 49 of the first connector 41 and the other of the planar sections of the second connector 61 (for example, the planar section 65) contact each other. In the present embodiment, two (a plurality of) contact portions (contact surfaces or contact points) located apart from each other about the rotation axis R are formed on the first connector 41 and the second connector 61.

Inside the fitting hole 55, a space 70 is formed between the proximal end of the first connector 41 and the bottom face 56 of the fitting hole 55. The proximal end of the first connector 41 is positioned between the proximal end and the distal end of the second connector 61, and the distal end of the second connector 61 is positioned between the proximal end and the distal end of the first connector 41. In the direction along the rotation axis R, the length (dimension) L1 of the first connector 41, the length (dimension) L2 of the second connector 61, the length (fitting length) L3 of the fitting portion 60, and the length L4 of the space 70 are defined. At this time, the fitting length L3 of the fitting portion 60 is smaller than the length L1 of the first connector 41 and the length L2 of the second connector 61. The rotary body 31 and the rotary body 51 are arranged so that the length L4 of the space 70 in the direction along the rotation axis R is equal to or greater than a predetermined length.

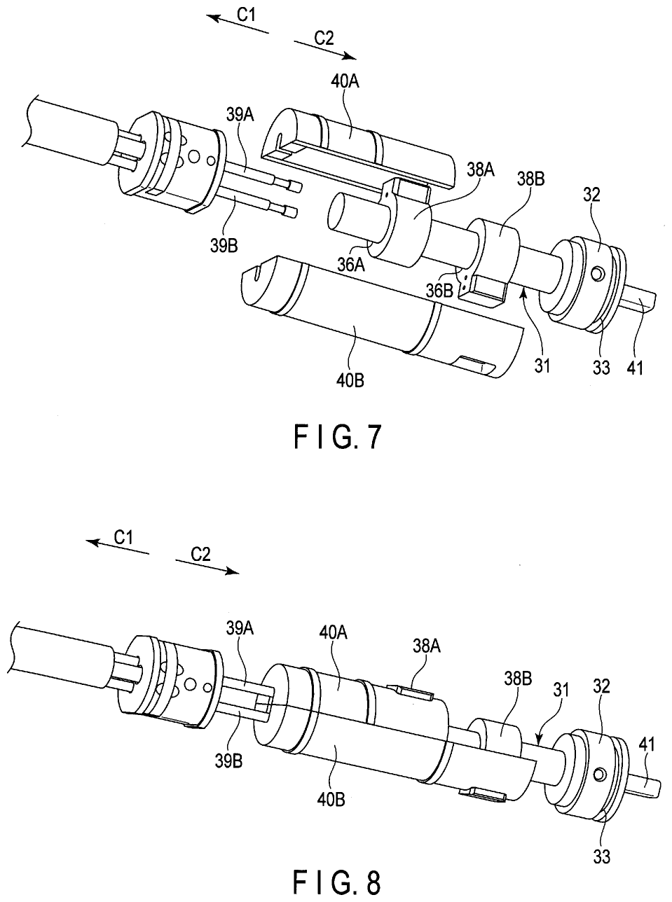

Next, a procedure of assembling the treatment instrument 1, which is the medical device of the present embodiment, will be described with reference to FIGS. 7 to 11. The procedure of assembling the treatment instrument 1 is an example of a method of manufacturing the treatment instrument 1 and a method of adjusting the treatment instrument 1. When assembling the treatment instrument 1, the holder 21 and the rotary base 26 are first mounted inside the housing 3. The operation dial 20 and the rotary body 51 are then mounted to the holder 21. One end of each of the wires 39A and 39B is connected to the end effector 7. The other end of each of the wires 39A and 39B are guided through the inside of the sheath 5 to the inside of the housing 3.

Next, the wire 39A is connected to the nut 38A mounted to the rotary body 31 via the relay member 40A, as shown in FIGS. 7 and 8. The wire 39B is connected to the nut 38B mounted to the rotary body 31 via the relay member 40B.

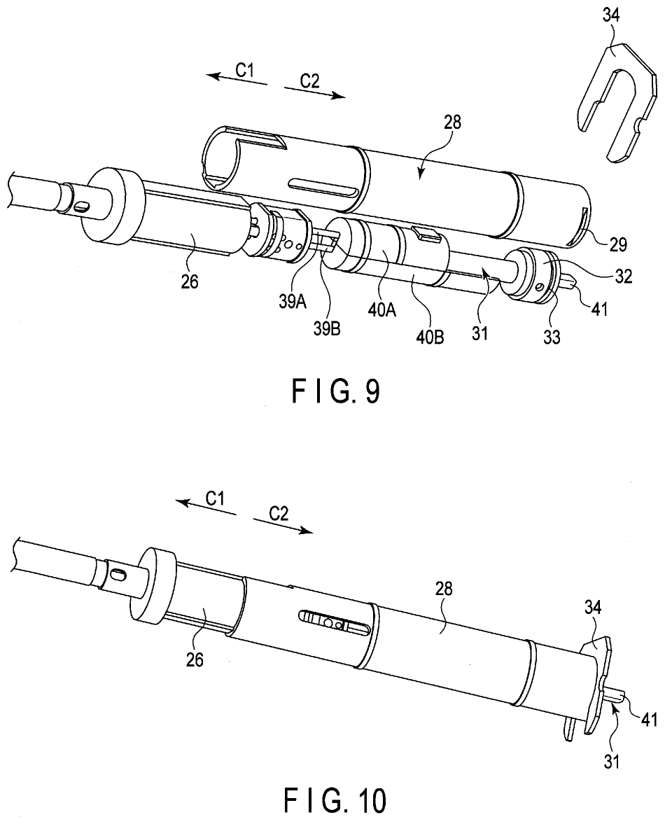

Next, the rotary body 31 to which the wires 39A and 39B are connected is inserted into the support member 28, as shown in FIGS. 9 and 10. The engaging member 34 is then mounted to the engaging groove 33 provided in the ring member 32 of the rotary body 31 and the slit 29 of the support member 28, so that the rotary body 31 is mounted inside the support member 28. In this state, the rotary body 31 is rotatable about the rotation axis R (longitudinal axis C) with respect to the support member 28, and the movement of the rotary body 31 in the direction along the rotation axis R (longitudinal axis C) relative to the support member 28 is regulated.

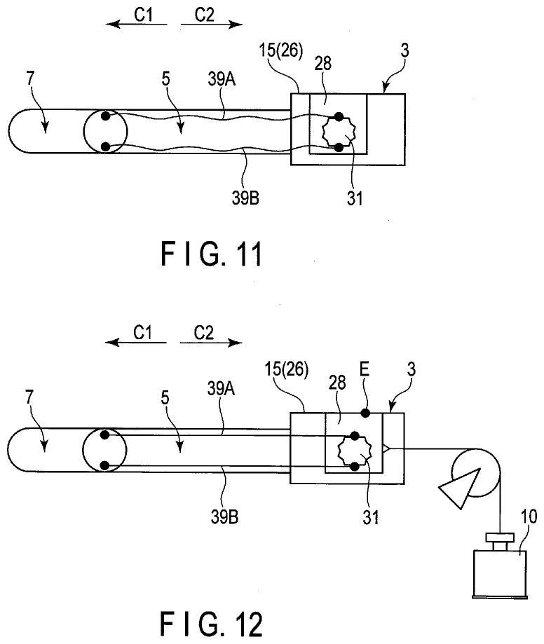

Next, the support member 28 is pulled to the proximal side with respect to the housing 3 by using a pulling device 10 such as a push-pull gauge, as shown in FIGS. 11 and 12. When the support member 28 is pulled to the proximal side, the rotary body 31 mounted inside the support member 28, the nuts 38A and 38B, and the wires 39A and 39B are pulled together with the support member 28 to the proximal side with respect to the housing 3 and the rotary base 26. At this time, the wire 39A and the wire 39B are pulled to both sides between the end effector 7 and the rotary body 31, so that a predetermined tension is applied to the wire 39A and the wire 39B.

The first connector 41 of the rotary body 31 is inserted from the distal side into the second connector 61 of the rotary body 51, and the first connector 41 and the second connector 61 are engaged with each other as described above. Thereby, the rotary body 31 and the rotary body 51 are connected to each other in a state where the rotational drive force is transmittable.

The support member 28 is bonded to the rotary base 26 with the rotary body 31 and the rotary body 51 engaged with each other and a predetermined tension applied to the wire 39A and the wire 39B. For example, laser welding, thermal caulking, or the like is used to bond the support member 28 to the rotary base 26. At this time, the support member 28 is fixed to the rotary base 26 at a bonding position E, for example. When the support member 28 is fixed to the rotary base 26 with a predetermined tension applied to the wires 39A and 39B, the wires 39A and 39B are mounted inside the housing 3 with the predetermined tension applied thereto.

In the present embodiment, the wires 39A and 39B are mounted inside the housing 3 with a predetermined tension applied thereto. Thereby, the wires 39A and 39B are mounted without loosening between the end effector 7 and the rotary body 31. As a result, even when an external force or the like is applied to the end effector 7, the wires 39A and 39B are prevented from moving relative to the sheath 5 and the housing 3, and the end effector 7 is prevented from unintentionally flexing or curving with respect to the sheath 5 due to the external force or the like.

In the present embodiment, the support member 28 for supporting the rotary body 31 to which the wires 39A and 39B are connected is provided. The support member 28 supports the rotary body 31 with the rotary body 31 being rotatable about the longitudinal axis C and the movement of the rotary body 31 in the longitudinal direction regulated. Therefore, when the support member 28 is mounted to the housing 3, the rotary body 31 can be mounted to the housing 3 with the rotary body 31 being rotatable with respect to the housing 3. Also, a pair of wires 39A and 39B are connected to the support member 28 via the rotary body 31. Therefore, when the support member 28 to which the pair of wires 39A and 39B are connected is connected to the housing 3, the pair of wires 39A and 39B can be connected to the housing 3 in a single step. This makes it unnecessary to mount each one of a plurality of wires (e.g., 39A, 39B) separately to the housing 3 with a predetermined tension applied thereto, allowing the plurality of wires (e.g., 39A, 39B) to be mounted to the housing 3 at one time. As a result, the working process is reduced and the labor associated with the assembling can be saved.

In the present embodiment, the support member 28 is fixed to the housing 3, so that a plurality of wires (e.g., 39A and 39B) can be mounted to the housing 3 at one time. Therefore, a plurality of wires (e.g., 39A and 39B) can be mounted to the treatment instrument 1 with the tension evenly applied thereto. Also, a plurality of wires (e.g., 39A, 39B) are mounted to the housing 3 at one time, so that the mounting positions of the wires (e.g., 39A and 39B) can be adjusted at one time.

When the wires 39A and 39B are mounted with a predetermined tension applied thereto, the position at which the support member 28 is connected to the housing 3 may be shifted from a predetermined position due to the individual difference in length of the wires 39A and 39B because of, for example, the difference in elongation of the wires 39A and 39B.

In the present embodiment, the space 70 is formed between the proximal end of the first connector 41 and the bottom face 56 of the fitting hole 55. The length L3 of the fitting portion 60 between the first connector 41 and the second connector 61 is smaller than the length L1 of the first connector 41 and the length L2 of the second connector 61. That is, the first connector 41 is provided to the rotary body 31 over a range larger than the part fitted to the second connector 61 of the rotary body 51. Also, the second connector 61 is provided to the rotary body 51 over a range larger than the part fitted to the first connector 41 of the rotary body 31. Therefore, even when the position at which the support member 28 is connected to the housing 3 is shifted due to the individual difference of the wires 39A and 39B, or the like, the connector 41 and the connector 61 are effectively engaged with each other when the position of the rotary body 31 relative to the rotary body 51 is shifted, because the connector 41 and the connector 61 are provided to the rotary body 31 and the rotary body 51 over a range larger than the fitting portion 60.

The configuration of the present embodiment is also applicable to a configuration in which one bar or the like is used as a drive member instead of the wires 39A and 39B. That is, as long as the configuration is such that a drive member is mounted to the housing 3 with the tension applied thereto, the same effect as the present embodiment can be obtained.

In the present embodiment, the first connector 41 is inserted into the second connector 61, so that the first connector 41 and the second connector 61 are engageable with each other; however, the present invention is not limited thereto. For example, the first connector 41 may be a groove extending along the rotation axis R (longitudinal axis C), and the second connector 61 may be formed to be insertable into the first connector 41. In this case as well, the length L3 of the fitting portion 60 is smaller than the length L1 of the first connector 41 and the length L2 of the second connector 61.

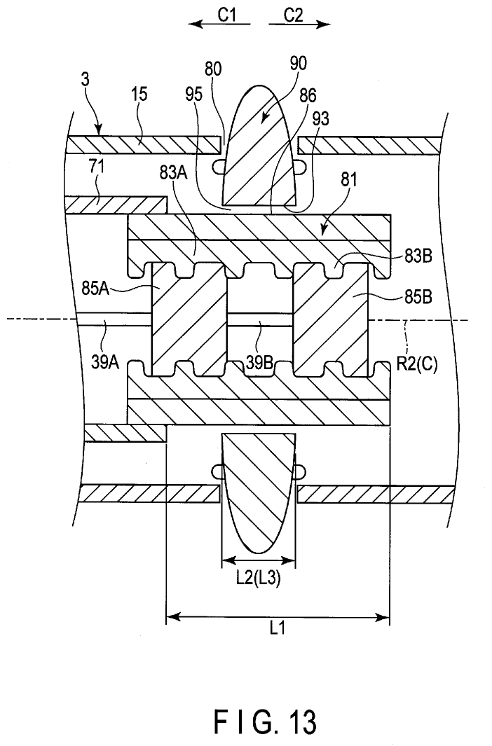

Another exemplary embodiment will be described with reference to FIG. 13. As shown in FIG. 13, the treatment instrument 1 of the present embodiment is provided with a support member 71, a rotary body (first rotor) 81, an operation dial (second rotor) 90, a pair of moving members 85A and 85B, and the pair of wires 39A and 39B.

The support member 71 is provided inside the housing 3. The support member 71 is a tubular member extending along the longitudinal axis C. The support member 71 is bonded to the inside of the housing 3 and thereby fixed to the housing 3.

The rotary body 81 is provided inside the housing 3. The rotary member 81 extends along a rotation axis R2. In the present embodiment, the rotation axis R2 of the rotary body 81 is coincident or approximately coincident with the longitudinal axis C. The rotary body 81 is formed in a tubular shape. The rotary body 81 is mounted to the proximal end of the support member 71. The rotary body 81 is rotatable about the rotation axis R2 with respect to the support member 71. Also, the movement of the rotary body 81 in a direction along the rotation axis R2 relative to the support member 71 is regulated.

The rotary body 81 includes a first connector 86. The first connector 86 forms a proximal end of the rotary body 81. The cross section of the first connector 86 that intersects (is perpendicular or approximately perpendicular to) the longitudinal axis C has an irregular shape (first irregular shape). In the present embodiment, the first connector 86 is formed by the outer peripheral surface of the rotary body 81.

The operation dial (operation member) 90 is mounted to the housing main body 15. The operation dial 90 is formed in a ring shape extending about the rotation axis R2 (longitudinal axis C) (in the circumferential direction). The operation dial 90 is rotatable about the rotation axis R2 (longitudinal axis C) with respect to the housing 3. At least a part of the outer peripheral surface of the operation dial 90 is exposed to the outside from an opening 80 provided to the housing main body 15. The operation dial 90 rotates about the rotation axis R2 with respect to the housing main body 15 when an operation input is performed to the part of the operation dial 90 exposed from the housing main body 15. An operation of flexing or curving the end effector 7 with respect to the sheath 5 is input to the operation dial 90.

The operation dial 90 includes a fitting hole 93 (second connector). The fitting hole 93 passes through the operation dial 90 along the rotation axis R2 (longitudinal axis C). The cross section of the fitting hole 93 that intersects (is perpendicular or approximately perpendicular to) the rotation axis R2 has an irregular shape (second irregular shape). The fitting hole 93 is formed in a shape that fits to the first connector 86. That is, the fitting hole 93 is formed in a shape complementary to the first connector 86. In the present embodiment, the second connector is formed by the inner peripheral surface of the fitting hole 93 formed in the operation dial 90.

In the present embodiment, the first connector 86 of the rotary body 81 is inserted into the fitting hole (second connector) 93 of the operation dial 90, so that the first connector 86 and the second connector 93 are engaged with each other to form a fitting portion 95. In the present embodiment, the entire length of the fitting hole (second connector) 93 becomes the fitting portion 95 in the direction along the rotation axis R2. By engaging the first connector 86 and the second connector 93, the rotation of the rotary body 81 about the rotation axis R2 with respect to the operation dial 90 is regulated, and a rotational drive force about the rotation axis R2 (longitudinal axis C) can be transmitted from the operation dial 90 to the rotary body 81. Therefore, when the operation dial 90 rotates about the longitudinal axis C (rotation axis R2), the rotational drive force is transmitted to the rotary body 81, and the rotary body 81 and the operation dial 90 rotate together about the longitudinal axis C (rotation axis R2) with respect to the housing main body 15.

In the direction along the rotation axis R2, the length (dimension) L1 of the first connector 86, the length (dimension) L2 of the second connector 93, and the length (fitting length) L3 of the fitting portion 95 are defined. The length L2 of the second connector 93 and the fitting length L3 of the fitting portion 95 are the same or approximately the same. The fitting length L3 of the fitting portion 95 is smaller than the length L1 of the first connector 86.

The rotary body 81 includes a first screw portion 83A and a second screw portion 83B. The first screw portion 83A and the second screw portion 83B are juxtaposed in the direction along the rotation axis R2. The first screw portion 83A and the second screw portion 83B have a spiral thread (female thread) formed on the inner peripheral surface of the rotary body 81. The first screw portion 83A and the second screw portion 83B are formed to be reversed with respect to each other.

The first moving member 85A and the second moving member 85B are mounted to the rotary body 81. A thread (male thread) that is screwed into the first screw portion 83A is formed on the outer peripheral surface of the first moving member 85A. The first moving member 85A is screwed into the first screw portion 83A to thereby be mounted to the rotary body 81. One end of the first wire 39A is connected to the first moving member 85A. The other end of the first wire 39A is connected to the end effector 7.

A thread (male thread) that is screwed into the second screw portion 83B is formed on the outer peripheral surface of the second moving member 85B. The second moving member 85B is screwed into the second screw portion 83B to thereby be mounted to the rotary body 81. One end of the second wire 39B is connected to the second moving member 85B. The other end of the second wire 39B is connected to the end effector 7.

Each of the first moving member 85A and the second moving member 85B is movable in the direction along the rotation axis R2 with respect to the support member 71. The rotary body 81 is rotatable about the rotation axis R2 with respect to the first moving member 85A and the second moving member 85B. Each of the first moving member 85A and the second moving member 85B moves in the direction along the rotation axis R2 (longitudinal axis C) relative to the rotary body 81 when the rotary body 81 rotates about the rotation axis R2. At this time, the first moving member 85A and the second moving member 85B move to opposite sides with respect to each other in the direction along the rotation axis R2 (longitudinal direction).

With the configuration described above, when the operation dial 90 rotates about the longitudinal axis C (rotation axis R2) with respect to the housing main body 15, the rotational drive force is transmitted from the operation dial 90 to the rotary body 81, so that the rotary body 81 rotates about the longitudinal axis C (rotation axis R2) together with the operation dial 90. Then, the first moving member 85A and the second moving member 85B move to opposite sides with respect to each other, and the pair of wires 39A and 39B move to opposite sides with respect to each other. Thereby, the end effector 7 flexes or curves with respect to the sheath 5.

When assembling the treatment instrument 1, which is a medical device of the present embodiment, each of the wires 39A and 39B is connected between the end effector 7 and the rotary body 81. The rotary body 81 is mounted to the support member 71 with the movement of the rotary body 81 in the direction along the rotation axis R2 relative to the support member 71 regulated and the rotary body 81 rotatable about the rotation axis R2.

The support member 71 can be fixed to the housing 3 with the support member 71 pulled to the proximal side, so that the wires 39A and 39B can be mounted to the housing 3 with a single bonding with a predetermined tension evenly applied to both of the wires 39A and 39B.

Also, in the present embodiment, the length L3 of the fitting portion 95 between the first connector 86 and the second connector 93 is smaller than the length L1 of the first connector 86. That is, the first connector 86 is provided to the rotary body 81 over a range larger than the part fitted to the second connector 93 of the operation dial 90. Therefore, even when the position at which the support member 71 is connected to the housing 3 and the position of the rotary body 81 relative to the operation dial 90 are shifted due to the individual difference of the wires 39A and 39B, or the like, the first connector 86 and the second connector 93 are effectively engaged with each other.

Also, in the present embodiment, the first connector 86 is inserted into the second connector 93 so that the first connector 86 and the second connector 93 are engageable with each other; however, the present invention is not limited thereto. For example, the first connector 86 may be a groove provided at the proximal end of the rotary body 81, and the second connector 93 may be formed to be fitted into the first connector 86. In this case, the length L3 of the fitting portion 95 is smaller than the length L1 of the first connector 86 and the length L2 of the second connector 93. That is, the length L3 of the fitting portion 95 is smaller than at least one of the length L1 of the first connector 86 or the length L2 of the second connector 93.

(Common Configuration of Embodiments, etc.)

The medical device (1) includes: a sheath (5) with a distal end and a proximal end; an end effector (7) provided at the distal end of the sheath (5), the end effector (7) capable of flexing or curving with respect to the sheath (5); a drive member (39A, 39B) configured to move relative to the sheath (5) and thereby flex or curve the end effector (7) with respect to the sheath (5); a first rotor (31:81) configured to rotate about a rotation axis (R) with respect to the drive member (39A, 39B) to thereby transmit a drive force to the drive member (39A, 39B) and move the drive member (39A, 39B) relative to the sheath (5); a first connector (41:86) provided to the first rotor (31:81), a cross section of the first connector (41:86) that intersects the rotation axis (R) having a first irregular shape; a second rotor (51:90) connected to the first rotor (31:81) so as to be rotatable about the rotation axis (R) together with the first rotor (31:81); and a second connector (61:95) provided to the second rotor (51:90), a cross section of the second connector (61:95) that intersects the rotation axis (R) having a second irregular shape, the second connector (61:95) fitted to the first connector (41:86), and the second connector (61:95) configured to transmit a drive force for rotating the first rotor (31:81) to the first rotor via the first connector (41:86) when the second rotor (51:90) rotates about the rotation axis (R); wherein a length of a fitting portion between the first connector (41:86) and the second connector (61:95) is smaller than at least one of a length of the first connector (41:86) and a length of the second connector (61:95).

The present invention is not limited to the above embodiments, and can be modified in various manners in practice when implementing the invention without departing from the gist of the invention. The respective embodiments may be appropriately combined to the extent possible, in which case a combined effect will be obtained. Furthermore, the above embodiments include various stages of invention, and various inventions can be derived by appropriately combining the disclosed elements.

1. A medical device comprising:
a sheath with a distal end and a proximal end;
a housing provided at the proximal end of the sheath;
an end effector provided at the distal end of the sheath, the end effector configured to bend with respect to the sheath;
a drive member including a first wire configured to move relative to the sheath and cause the end effector to bend with respect to the sheath; and
a support member mounted inside the housing, the drive member being mounted inside the support member.

2. The medical device according to claim 1, wherein the support member is mounted to the housing and configured to apply a predetermined tension to the drive member.

3. The medical device according to claim 1, further comprising a first rotor mounted on the support member, wherein:
the drive member further includes a second wire, the first wire and the second wire being connected to the first rotor.

4. The medical device according to claim 1, further comprising a flexural drive unit that includes the drive member and a first rotor provided along a longitudinal axis, wherein:
the drive member is configured to move in a first direction along the longitudinal axis when the first rotor rotates about the longitudinal axis.

5. The medical device according to claim 4, wherein:
the drive member further includes the first wire and a second wire;
the first rotor is provided with a first nut and a second nut; and
the first wire is connected to the first nut and the second wire is connected to the second nut.

6. The medical device according to claim 5, wherein:
the first rotor includes a first screw portion with a first thread and a second screw portion with a second thread;
the second thread being reversed with respect to the first thread;
the first screw portion is provided with the first nut; and
the second screw portion is provided with the second nut.

7. The medical device according to claim 6, wherein the first wire and the second wire are configured to move to opposite sides with respect to each other along the longitudinal axis when the first rotor rotates about the longitudinal axis, and when the first nut and the second nut move to opposite sides with respect to each other.

8. The medical device according to claim 4, further comprising: a second rotor having a second connector, wherein:
the first rotor has a first connector that is configured to connect to the second connector; and
a space is formed between a proximal end of the first connector and the second connector.

9. The medical device according to claim 1, wherein the end effector is supplied with energy to thereby treat a treatment target.

10. The medical device according to claim 9, wherein:
the end effector is formed of a pair of grasping pieces configured to open and close; and
the housing includes a grip and a movable handle configured to open and close with respect to the grip to open and close the end effector.

11. The medical device according to claim 10, wherein the housing is provided with an operation dial configured to cause the end effector to flex or curve by moving a flexural drive unit.

12. A method of manufacturing a medical device, the method comprising:
connecting a drive member to an end effector;
guiding the drive member through a sheath to a housing provided at a proximal end of the sheath;
mounting a part of a flexural drive unit including the drive member to a support member;
pulling the support member to thereby apply a predetermined tension to the drive member; and
mounting the support member to an inside of the housing.

13. The method of manufacturing a medical device according to claim 12, wherein the drive member is a wire.

14. The method of manufacturing a medical device according to claim 13, wherein:
two or more of wires are employed; and
the support member is fixed inside the housing, thereby allowing the two or more of the wires to be mounted at one time.

15. The method of manufacturing a medical device according to claim 12, wherein a laser welding or a thermal caulking is used to mount the support member inside the housing.

16. A method of adjusting a medical device which is configured to flex or curve an end effector provided at a distal end of a sheath by using a wire, the method comprising:
mounting two or more of the wires to a support member;
pulling the support member to thereby apply a predetermined tension to the two or more of the wires; and
mounting the support member inside a housing provided at a proximal end of the sheath with the tension applied, thereby adjusting mounting positions of the wires at one time.
